(19) United States
(12) Patent Application Publication
(10) Pub. No.: US 2007/0184117 A1
(43) Pub. Date: Aug. 9, 2007

(54) Tocopheryl polyethylene glycol succinate powder and process for preparing same

(76) Inventors: Stephen Gregory, Tynygongl (GB); Bruce Colin Jones, Llangefni (GB); Andy Hugh Singleton, Kingsport, TN (US); Wendy Anne Johnson, Capelle aan den ljssel (NL)

Correspondence Address: William K. McGreevey;Eastman Chemical Company, P.O. Box 511, Kingsport, TN, 37662-5075, US

(21) Appl. No.: 11/493,215

(22) Filed: Jul. 26, 2006

Related U.S. Application Data
(60) Provisional application No. 60/705,057, filed on Aug. 3, 2005.

(51) Int. Cl. A61K 9/14

(52) U.S. Cl. 424/489

(57) ABSTRACT

A powdered tocopheryl polyethylene glycol succinate (TPGS™) having an average particle size of less than about 1000 microns. In one embodiment, the powdered tocopheryl polyethylene glycol succinate is prepared by a process that includes atomizing a fluidic tocopheryl polyethylene glycol succinate into an environment suitable for solidifying the atomized tocopheryl polyethylene glycol succinate. In another embodiment, the powdered tocopheryl polyethylene glycol succinate is prepared by a process of applying a force to a solid tocopheryl polyethylene glycol succinate starting material that is sufficient to produce a powdered product.

This application claims the priority benefit of provisional application No. 60/705,057, filed on Aug. 3, 2005, incorporated by reference herein.

### BACKGROUND OF THE INVENTION

The present invention relates to tocopheryl polyethylene glycol succinate powder and methods for making the same.

Tocopheryl polyethylene glycol succinate has been used in a variety of food and pharmaceutical formulations and is generally recognized as safe for such uses. Generally, tocopheryl polyethylene glycol succinate, available from Eastman Chemical Company under the tradename Vitamin E TPGS™, is a water-soluble preparation of a fat-soluble vitamin and is disclosed in greater detail in U.S. Pat. Nos. 3,102,078, issued to Robeson on Aug. 27, 1963 and 2,680,749 issued to Cawley et al. on Jun. 8, 1954, the entire disclosures of which is incorporated herein by reference. The polyoxyethylene glycol moiety of the Vitamin E TPGS™ has a molecular weight in the range of about 200 to 20,000, desirably of about 400 to 10,000, preferably of about 400 to 3000, and more preferably from about 400 to 2000 and most preferably the water-soluble preparation of a fat-soluble vitamin is Vitamin E succinate polyethylene glycol 1000. The commercial product is prepared by esterifying the carboxyl group of crystalline d-α-tocopheryl acid succinate (or the d,l-form in the case of synthetic vitamin E) with polyethylene glycol 1000.

At room temperature Vitamin E TPGS™ is a waxy low melting solid and typically is sold in containers in the form of a solid block. Accordingly, to use the TPGS™ the entire container is heated to a temperature above the melting temperature, from about 37 to 41° C. and the desired amount is poured out. Although TPGS™ is heat-stable having a decomposition temperature of about 200° C., it is inconvenient for the user to melt all the TPGS™ in the container for each use. Repeated heating and cooling cycles of the material can cause discoloration and may result in a decreased shelf life for the TPGS™.

Alternatively, the desired amount of TPGS™ can be removed from the container by breaking the solid cake into pieces. However, this means of removing the TPGS™ is inconvenient and can increase the risk of product contamination. Furthermore, it is hard to be quantitative in removing a specific amount from a waxy solid block.

Accordingly, there is a need for a solid the TPGS™ that can be stored under atmospheric conditions, yet easily measured and incorporated into a final product without resorting to the cumbersome methods described above.

### SUMMARY OF THE INVENTION

The present invention is a TPGS™ powder that can be stored under atmospheric conditions of temperature, pressure and humidity without compromising the handling characteristics of the powder. Accordingly, the present invention is a TPGS™ powder having an average particle size of less than about 1000 microns.

The present invention is also directed toward a method of making a powdered TPGS™ having an average particle size of less than about 1000 microns. In one embodiment the process includes atomizing fluidic TPGS™ into an environment suitable for solidifying the atomized TPGS™. In a second embodiment the process includes cooling solid TPGS™ in an appropriate apparatus sufficiently to embrittle the solid TPGS™, and applying a force to the brittle TPGS™ sufficient to form a powder.

It is an object of the present invention to provide a TPGS™ powder having an average particle size of less than about 1000 microns.

It is another object of the present invention to provide a process for making a TPGS™ powder having an average particle size of less than about 1000 microns.

These and other objects and advantages of the present invention will become more apparent to those skilled in the art in view of the following description. It is to be understood that the inventive concept is not to be considered limited to the constructions disclosed herein but instead by the scope of the appended claims.

### DETAILED DESCRIPTION OF THE INVENTION

The TPGS™ powder is a small solid particle and can have a surface tackiness such that the powder particles do not stick together significantly to cause a problem in handling and pouring of the TPGS™. The surface tackiness is preferably no greater than 1500 grams and most preferably no greater than about 1485 grams. The powder form of TPGS™ can allow for improved handling of TPGS™, including improved pourability due to the flow of a powder and can allow for broader uses, such as being directly compressible into forms such as tablets in pharmaceutical applications.

As discussed briefly above, TPGS™ can be prepared by esterifying tocopheryl acid succinate with polyethylene glycol (PEG). The esterification procedure is preferably performed in a solvent media and may be promoted by any well known esterification catalyst. The polyethylene glycol used to esterify the tocopheryl acid succinate desirably has a number average molecular weight ranging from about 200 to about 20,000, desirably of from about 400 to about 10,000, preferably from about 400 to about 3000, and more preferably from about 400 to about 2000 and most preferably the polyethylene glycol has a number average molecular weight of about 1000. The resulting product comprises at least polyethylene glycol esters of tocopheryl acid succinate. The esters can comprise, as the major component, mono-ester tocopheryl polyethylene glycol succinate, and di-esters of tocopheryl polyethylene glycol succinate.

Desirably, the powder TPGS™ particle size is such that the powder is flowable or pourable so that the powder can be easily handled, such as pouring, weighing or measuring out the desired quantity. Desirably, the size of the powder particles weigh equal to or less than about 1 gram. In a preferred embodiment the TPGS™ powder has an average particle weight from about 10 mg to about 150 mg, preferably from about 15 mg to about 90 mg, and most preferably from about 20 mg to about 80 mg. The powder TPGS™ has an average particle size no greater than about 1000 microns and preferably no greater than about 500 microns and most preferably no greater than about 260 microns. The powder has a surface tackiness of no greater than about 1500 grams, preferably no greater than about 1000 grams and most preferably no greater than about 550 grams.

In one embodiment, the powder TPGS™ is prepared by fluidizing solid TPGS™ to form a liquid or fluidic state; and atomizing the liquid TPGS™ to form liquid droplets of the size described above wherein the atomizing TPGS™ is sprayed into an environment that is suitable for solidifying the atomized, fluidic TPGS™ to form a powder. In a preferred embodiment, the powder TPGS™ is recovered and collected using techniques and apparatus known to those skilled in the art.

In one embodiment fluidic TPGS™ is prepared by heating solid TPGS™ to a temperature of from about 40° C. to about 85° C., preferably from about 45° C. to about 75° C., and most preferably a range from about 45° C. to about 55° C. In another embodiment, fluidic TPGS™ is prepared by dissolving solid TPGS™ using an appropriate solvent, such as acetone, methyl-ethyl ketone, methanol, ethanol, propanol, methylene chloride and mixtures thereof. Desirably, the fluidic TPGS™ has a viscosity from about 20 to 5000 centi-poise/sec (cps), preferably less than about 1000 cps and more preferably less than 500 cps.

To form the powdered TPGS™, the fluidic TPGS™ can, for example, be atomized into substantially predetermined and appropriately sized droplets. Conventional equipment may be used in atomizing the fluidic TPGS™. For example, the fluidic TPGS™ can be sprayed or forced through a nozzle or orifice, with or without a fluid carrier, such as air, nitrogen, or other non-reactive or inert material which atomizes the fluidic TPGS™. Such atomizing equipment is well known to those skilled in the art.

The atomized TPGS™ can be sprayed into a solidifying environment that is suitable for allowing the atomized TPGS™ to solidify into a powder. Equipment suitable for such phase conversion includes, but is not limited to, a co-current and/or counter-current spray drying vessels. As used herein, the term “co-current” means that the atomized TPGS™ is solidified in a direction substantially parallel to the spray stream exiting the spray nozzle or orifice and preferably, is solidified in a direction that is less than about 45 degrees relative to the spray stream exiting the spray nozzle. As used herein, the term “counter-current” means that the atomized TPGS™ is solidified in a direction that is at an angle greater than about 45 degrees relative to the spray stream exiting the spray nozzle. In some embodiments such counter-current spray drying vessels have a spray direction that is about 180 degrees opposite the direction of the atomized particle solidification direction. Additionally, the spray drying vessel may optionally utilize an inert carrier gas stream to assist in the solidification of the fluidic TPGS™, particle distribution of the atomized TPGS™ in the vessel and/or removal of the powdered TPGS™ from the spray drying vessel. Such co-current and counter-current spray drying equipment is well known in the art.

The spray drying vessel desirably is operated at conditions of temperature and pressure below the melting point of the TPGS™. The atomized TPGS™ has a residence time in the solidifying environment that is sufficient to allow the fluidic TPGS™ to solidify sufficiently to substantially prevent agglomeration. As will be understood by those skilled in the art, the residence time is dependent on the temperature of the environment in which it is sprayed, the amount and type of solvent used, and the type and temperature of the carrier gas, if used. Non-limiting examples of useful equipment are available from Niro Ltd., 1 The Quadrant, Abingdon Science Park, Abingdon, Oxon. OX14, 3YS, United Kingdom, and Invensys APV, 395 Fillmore Avenue, Tonawanda, N.Y. 14150, USA. Typically the spray drying vessel is operated at a temperature of less than about 31° C. and a pressure of about less than about 50 bar (5000 kPa). The atomized TPGS™ can have a residence time in the solidifying environment of from about 1 second to about 5 minutes.

In a second embodiment of the method, powdered TPGS™ can be prepared directly from solid TPGS™ by applying a force to, or otherwise physically processing a solid TPGS™ starting material that is sufficient to produce a powdered product. Desirably, the solid TPGS™ starting material is ground or milled to the desired particle size. The solid TPGS™ material should be at a temperature that is less than about 31° C. and preferably, less than about 0° C. to ensure that the TPGS™ remains in a solid phase during the grinding or milling operation.

Examples of useful milling equipment include a Spex Freezer Mill available from Spex Industries, Inc., Metuchen, N.J., USA, and an air mill known to those skilled in the art.

In some embodiments the powdered TPGS™ is directly compressible. The direct compressibility allows the TPGS™ powder to be directly compressed into a tablet form without further processing.

The present invention is illustrated in greater detail by the specific examples presented below. It is to be understood that these examples are illustrative embodiments and are not intended to be limiting of the invention, but rather are to be construed broadly within the scope and content of the appended claims. All parts and percentages in the examples are on a weight basis unless otherwise stated.

The following test procedures were used in evaluating the analytical properties of the products herein.

Differential scanning calorimetry (DSC) was used for determining the melting temperature of TPGS™. The instrument used was a Mettler differential scanning calorimeter (Model 821, Mettler Toledo Inc., Columbus, Ohio). A TPGS™ sample of about 4.8 mg was weighed and placed on a 40 micrometer pan and hermetically sealed. The heating and cooling cycles were set between −140° C. and 85° C. with a 20° C./min heating rate. Cooling was done by liquid nitrogen purge (30 ml/min.) at temperatures from about 15° C. to about minus 130° C., for 10 minutes isothermally at minus 130° C., then heating to 75° C., held for 1 minute and then cooled back down to minus 130° C. and held isothermally for 10 minutes. A second cycle was then run from minus 130° C. to 75° C. All the cycles had a heating and cooling rate of 20° C./minute. The melting temperature of TPGS™ was then determined by the temperature at which abrupt changes of heat absorption curve occurred.

The compositions of TPGS™ were determined by an HPLC method using the following typical conditions.

### EXAMPLE 1

This example illustrates a method for preparing a powdered TPGS™ from solid material. A Spex Freezer/Mill was used to cryogenically grind Eastman Vitamin E TPGS™ 1000, NF. The objective was to determine the range of particles formed by cryo grinding.

The Freezer/Mill chamber was filled with liquid nitrogen. Five grams of flaked Vitamin E TPGS™ 1000, NF were weighed into a sample tube. A metal rod, used as an impactor, was placed in the sample tube with the flaked TPGS™ and the tube was sealed. The sample was placed in the chamber and the latch was closed. The vapor stream was allowed to decrease for approximately four minutes and the timer was set for a six minute run time. The sample was removed from the chamber after six minutes and allowed to warm to room temperature. The TPGS™ was removed from the sample tube and submitted for particle size analysis. Primary particles were blue with the smallest being about 0.5 microns.

### EXAMPLE 2

This example illustrates a method for preparing a powdered TPGS™ from a fluidized material. One hundred and seventy-three (173) grams of melted TPGS™ at a temperature of 75° C. were added to 300 grams of acetone. The solution was mixed until the TPGS™ was in solution. The sample was spray dried using an APV Anhydro Lab Model 1 spray dryer. Atomization was accomplished using a two-fluid nozzle with nitrogen as the atomizing gas. The solution was fed to the dryer using a Masterflex tubing pump. The conditions are specified in Table 1 below.

TABLE 1

| | |
|---|---|
| Inlet Temperature ° C. | 23 |
| Outlet Temperature ° C. | 20 |
| Nitrogen delta P (inches of water) | 60 |
| Atomization Pressure (psi) | 45 |
| Pump Speed | 18 |
| Feed Wt (g) | 304 |
| Run Time (min.) | 28 |
| Feed Rate (g/min.) | 10.8 |
| Yield (g) | 13.5 |

Due to the low melting point of vitamin E TPGS™, no heat was used. The average particle size of the spray dried TPGS™ ranged from about 1 to about 60 microns.

The Tm and Tg of the TPGS™ powder were determined to be 38.4° C. and −58.3° C., respectively. The analysis was conducted using a TA Instruments DSC 2920. The sample was heated from −75° C. to 75° C. at a rate of 20° C. per minute in nitrogen.

The oxidative degradation onset point was determined to be about 166.1° C. with its exothermic peak temperature being about 193.8° C. The analysis was conducted in air using a TA Instruments High Pressure DSC 912. The sample was heated from 25° C. to 300° C. using a scanning rate of 10° C./min. in oxygen @ 550 psi.

Surprisingly, aqueous solutions can be readily prepared from the powdered TPGS™ using chilled water, room temperature water, or heated water. Generally, solutions prepared using the wax form of TPGS require that the wax and water phase be heated above the Tm of Vitamin E TPGS, which is about 40° C.

### EXAMPLE 3

A twenty percent solution of powdered TPGS in water was prepared. Twenty grams of powdered TPGS™ were added to eighty grams of 5° C. Millipore water with mixing. The TPGS™ was added in four gram aliquots and mixed until in solution.

### EXAMPLE 4

A twenty percent solution of powdered TPGS™ in water was prepared. Twenty grams of powdered TPGS™ were added to eighty grams of 24° C. Millipore water with mixing. The TPGS™ was added in four gram aliquots and mixed until in solution.

### EXAMPLE 5

A twenty percent solution of powdered TPGS™ in water was prepared. Twenty grams of powdered TPGS™ were added to eighty grams of 70° C. Millipore water with mixing. The TPGS™ was added in four gram aliquots and mixed until in solution

Having described the invention in detail, those skilled in the art will appreciate that modifications may be made to the various aspects of the invention without departing from the scope and spirit of the invention disclosed and described herein. It is, therefore, not intended that the scope of the invention be limited to the specific embodiments illustrated and described but rather it is intended that the scope of the present invention be determined by the appended claims and their equivalents. Moreover, all patents, patent applications, publications, and literature references presented herein are incorporated by reference in their entirety for any disclosure pertinent to the practice of this invention.

What is claimed is:

1. A composition comprising a powdered tocopheryl polyethylene glycol succinate wherein the tocopheryl polyethylene glycol succinate has a polyethylene glycol moiety having a number average molecular weight ranging from about 200 to about 20,000 and the powdered tocopheryl polyethylene glycol succinate has an average particle size of less than about 1000 microns.

2. The composition of claim 1 wherein the polyethylene glycol moiety has a number average molecular weight ranging from about 400 to about 10,000.

3. The composition of claim 1 wherein the polyethylene glycol moiety has a number average molecular weight ranging from about 400 to about 3000.

4. The composition of claim 1 wherein the polyethylene glycol moiety has a number average molecular weight ranging from about 400 to about 2000.

5. The composition of claim 1 wherein the polyethylene glycol moiety has a number average molecular weight of about 1000.

6. A method for preparing a powdered tocopheryl polyethylene glycol succinate comprising atomizing a fluidic tocopheryl polyethylene glycol succinate into an environment suitable for solidifying the atomized tocopheryl polyethylene glycol succinate.

7. The method of claim 6 further comprising heating a solid tocopheryl polyethylene glycol succinate to a temperature of from about 40° C. to about 85° C. to form the fluidic tocopheryl polyethylene glycol succinate.

8. The method of claim 7 wherein the solid tocopheryl polyethylene glycol succinate to a temperature of from about 45° C. to about 55° C.

9. The method of claim 6 further comprising dissolving a solid tocopheryl polyethylene glycol succinate in a solvent to form the fluidic tocopheryl polyethylene glycol succinate.

10. The method of claim 9 wherein the solvent is selected from the group consisting of acetone, methyl-ethyl ketone, methanol, ethanol, propanol, methylene chloride and mixtures thereof.

11. The method of claim 6 wherein the environment suitable for solidifying the atomized tocopheryl polyethylene glycol succinate is a spray drying vessel operated at conditions of temperature and pressure below the melting point of the tocopheryl polyethylene glycol succinate.

12. The method of claim 11 wherein the spray drying vessel is operated at a temperature of less than about 31° C. and at a pressure of about less than about 50 bar (5000 kPa).

13. A method for preparing a powdered tocopheryl polyethylene glycol succinate composition comprising physically processing a solid tocopheryl polyethylene glycol succinate starting material in a manner that is effective to produce a powdered product.

14. The method of claim 13 wherein the solid tocopheryl polyethylene glycol succinate starting material is subjected to grinding to produce the desired particle size.

15. The method of claim 14 wherein the grinding force is solid impacting member.

16. The method of claim 13 wherein the solid tocopheryl polyethylene glycol succinate is air milled to produce the desired particle size.
